(19) United States
(12) Patent Application Publication
(10) Pub. No.: US 2010/0279976 A1
(43) Pub. Date: Nov. 4, 2010

(54) USE OF ARTEMISININ AND ITS DERIVATIVES IN CANCER THERAPY

(75) Inventors: Hui Wang, Shanghai (CN); Tao Chen, Shanghai (CN); Junmei Hou, Shanghai (CN); Mian Li, Shanghai (CN)

Correspondence Address: OSHA LIANG L.L.P., TWO HOUSTON CENTER, 909 FANNIN, SUITE 3500, HOUSTON, TX, 77010, US

(73) Assignee: SHANGHAI INSTITUTES FOR BIOLOGICAL SCIENCES, CAS, Shanghai (CN)

(21) Appl. No.: 12/769,263

(22) Filed: Apr. 28, 2010

Related U.S. Application Data
(60) Provisional application No. 61/174,286, filed on Apr. 30, 2009.

(51) Int. Cl. A61K 31/7068; A61K 31/366; A61P 35/00; A61P 15/00; A61P 1/16

(52) U.S. Cl. 514/49; 514/450

(57) ABSTRACT

A method for treating cancer in a mammal includes administering to the mammal in need thereof a therapeutically effective amount of artemisinin (ART) or its derivative, such as dihydroartemisinin (DHA), artemether (ARM), or artesunate (ARS) alone or in combination with a chemotherapeutic agent, such as gemcitabine and carboplatin. A method for inhibiting tumor cell proliferation includes contacting a tumor cell with ART or its derivative, such as DHA, ARM, and ARS, in an amount effective to inhibit tumor cell proliferation or in combination with a chemotherapeutic agent, such as gemcitabine and carboplatin.

### CROSS-REFERENCE TO RELATED APPLICATIONS

This Application claims the benefit of U.S. Provisional Application 61/174,286 filed on Apr. 30, 2009, the disclosure of which is incorporated by reference in its entirety.

### BACKGROUND OF INVENTION

1. Field of the Invention

The invention relates generally to methods of treating cancers.

2. Background Art

Cancers remain a major health threat despite immense efforts in the search for cures. For example, human hepatocellular carcinoma (HCC) is one of the leading causes of cancer-related death worldwide, and more than 80% of liver cancer cases occur in developing countries, such as China and Africa. HCC has a long latency; therefore, it is often diagnosed at late stages when tumors are of high grade and progress rapidly. These characteristics, coupled with its high likelihood of invasion, lead to a poor prognosis for patients diagnosed with the disease. Nonsurgical approaches are necessary because patients with large tumors (>5 cm in diameter) or numerous lesions (>3) typically are not suitable for hepatic resection. Unfortunately, the activity of single chemotherapeutic agents is limited, with a very low response rate. Moreover, aggressive combination chemotherapeutic regimens have not led to any remarkable improvement in response rates. In advanced HCC, cancer cells do not respond to the cytotoxic effects of most of the available chemotherapeutic agents. Therefore, there is a pressing need to identify alternative chemotherapeutic strategies that circumvent these limitations.

Similarly, ovarian cancer poses a major health problem in women worldwide and is the fourth leading cause of cancer death in women in the United States. The 5-year survival rate for early-stage patients is 80-90%, but only 25% for those diagnosed at advanced stages of the disease. Unfortunately, most ovarian cancer patients have advanced disease at diagnosis. Although the ovarian cancer mortality rate has not changed significantly during the past few decades, the length of survival for patients has been steadily improving, largely as a result of clinical applications of newer and more effective chemotherapeutic drugs for adjuvant therapy after surgery. For instance, carboplatin (CBP) is one of the most important chemotherapeutic drugs used for adjuvant treatment of primary ovarian cancer and for metastatic disease. Its major mechanism involves the formation of DNA adducts, resulting in G2 phase cell cycle arrest, subsequently triggering apoptosis. While effective, CBP induces side effects, including neurotoxicity and nephrotoxicity.

Various other cancers are in similar states as HCC and ovarian cancer—i.e., there have been some progress in the treatments or prevention; however, more effective treatments are still needed. Therefore, there remains a continued need for novel drugs that can be used alone or in combination with conventional agents to overcome acquired drug resistance or sensitize tumors to therapy.

Phytochemicals show promise in cancer therapy because of their potential as chemopreventive agents and their chemotherapeutic activities. For example, phytochemicals have been found to be effective against HCC in experimental studies. Recently, gemcitabine, a novel nucleoside analogue that has a broad spectrum of antitumor activity in solid tumors, has been evaluated in clinical trials to treat HCC. Gemcitabine monotherapy improves the results of HCC treatment, as the reported median survival time increases up to 34 weeks. Because gemcitabine is particularly promising because of its low apparent toxicity profile, further studies in combination with other active agents are warranted.

FIG. 1A shows a chemical structure of artemisinin (ART), a natural product isolated from the plant Artemesia annua L. ART is widely used as an anti-malarial drug.

Various derivatives of ART, such as dihydroartemisinin (DHA), artemether (ARM), and artesunate (ARS) (FIG. 1A), also have potent activities against malarial parasites.

### SUMMARY OF INVENTION

The present invention relates to combination therapy for treating human cancers regardless the p53 status in tumors. Embodiments of this invention include a use of ART and its various derivatives (DHA, ARM, and ARS) alone or combined with other chemotherapeutic agents for treating human cancers. For example, ART and its derivatives may be combined with gemcitabine for treating hepatoma or combined with carboplatin for treating ovarian cancer.

In one aspect, the present invention relates to methods for treating cancer in a mammal. The methods may include administering to a mammal in need thereof a therapeutically effective amount of ART or its derivative, such as DHA, ARM, and ARS, alone or in combination with a chemotherapeutic agent.

In another aspect, the present invention relates to methods for inhibiting tumor cell proliferation. The methods may include contacting a tumor cell with ART or its derivative, such as DHA, ARM, and ARS, alone or in combination with a chemotherapeutic agent.

Other aspects and advantages of the invention will be apparent from the following description and the appended claims.

### BRIEF DESCRIPTION OF DRAWINGS

FIG. 1: A, chemical structures of the four ART compounds: ART, DHA, ARM, and ARS. B, Cytotoxicity of the four ART compounds to human hepatoma cells HepG2 (B1), Hep3B (B2), Huh-7 (B3), and BEL-7404 (B4) and normal human liver 7702 cells (B5). Cells are exposed to various concentrations of the compounds (0, 1, 5, 10, 25, 50, and 100 μmol/L) for 48 h followed by MTT assay. All assays are done in triplicate.

FIG. 2: The inhibitory effects of ART compounds on the growth of human hepatoma cells. HepG2 (A) and Hep3B (B) cells are exposed to 10 μmol/L ART and DHA alone or in combination with 10 μg/L gemcitabine for various durations (0, 24, 48, 72, and 96 h) followed by the MTT assay. All assays were done in triplicate.

FIG. 3: Effects of ART and DHA on cell cycle progression of human hepatoma cells. HepG2 (A) and Hep3B (B) cells are exposed to various concentrations of the compounds (0, 1, 10, 25, and 50 μmol/L) for 48 h followed by cell cycle distribution assay. All assays are done in triplicate. *, P<0.05 versus control; **, P<0.01 versus control. The effects of the compounds on the expression of cell cycle-related proteins are determined by Western blot analyses after HepG2 (C) and Hep3B (D) cells are exposed to various concentrations (5, 25, and 50 μmol/L) of the compounds for 48 h.

FIG. 4: Induction of apoptosis in human hepatoma cells HepG2 (A) and Hep3B (B). Cells are exposed to various concentrations of ART or DHA alone or in combination with gemcitabine for 48 h followed by apoptosis assay. All assays are done in triplicate. *, P<0.05 versus control; **, P<0.01 versus control. HepG2 (C) and Hep3B (D) cells are exposed to various concentrations of the compounds for 48 h, and the target proteins are detected by Western blot analyses.

FIG. 5: In vivo antitumor activity and effects on body weight of ART and DHA administered alone or in combination with gemcitabine to nude mice bearing HepG2 (A) and Hep3B (B) xenograft tumors. ART and DHA are given alone orally at doses of 50 and 100 mg/kg/d, 5 d/wk for 4 wk (A1, A2, B1, and B2), or in combination with gemcitabine (A3, A4, B3, and B4). Gemcitabine (80 mg/kg) is given on days 7, 11, and 15 by i.p. injection. Tumor mass may be determined by caliper measurement in two perpendicular diameters of the implant every 3 d. The toxic effects of administration of ART and DHA alone or in combination with gemcitabine on nude mice may be determined by recording the body weights of each mouse every 3 d throughout the experiment (A5, A6, B5, and B6). C and D, at the end of the treatment, tumor xenografts are removed, and proteins in the tumor homogenate are analyzed by Western blotting.

FIG. 6: A, the proposed mechanism(s) by which ART and/or DHA exert their effects via various proliferation- and apoptosis-related proteins. B, the proposed mechanism(s) by which ART and/or DHA enhance the therapeutic effects of gemcitabine.

FIG. 7: DHA selectively decreases cell viability and inhibits the growth of human ovarian carcinoma cells, but not non-tumorigenic ovarian surface epithelial cells. (A) Chemical structures of the four artemisinin (ARS) compounds; (B) Viability of human ovarian carcinoma cells (ovarian carcinoma A2780 and OVCAR-3) and non-tumorigenic OSE cells (IOSE144) after 48 hrs exposure to the ARS compounds as determined by MTT assay; C, Cell growth inhibition after 0, 24, 48 and 72 hrs exposure of A2780 and OVCAR-3 cells to DHA. Values are representative of at least three independent experiments with similar results, and are presented as the percentage of cell inhibition where vehicle-treated cells are regarded as 100% viable/0% growth inhibition.

FIG. 8: DHA induces dose-dependent apoptosis in human ovarian carcinoma cells. (A) Apoptosis in A2780 and OVCAR-3 cells treated with DHA (0, 5, 10, 25, 50 μM) for 24 hrs; (B) Data summary and analysis (*, P<0.001 versus the control, respectively). Data are representative of values from at least three independent experiments with similar results. The percentage of cellular apoptosis in control cells was regarded as 100%.

FIG. 9: Western blot analysis of protein expression levels indicates the effects of DHA on A2780 and OVCAR-3 ovarian cancer cell lines after 24 hrs exposure to specific doses of DHA.

FIG. 10: DHA causes disruption of the mitochondrial membrane potential and cytochrome c release. (A) and (B) Fluorescence (red and green) intensity values emitted by JC-1 fluorescent dye at specific excitation wave-lengths and the corresponding ratio of red/green (% of the control) after exposure to different concentrations of DHA for 24 hrs. Values are representative of at least three independent experiments with similar results (*, P<0.001 versus the control). (C) Western-blot analysis of the effects of 24 hrs DHA exposure (0, 5, 10, 25, 50 μM) on cytochrome c release from the mitochondria to the cytosol (mito-, mitochondrial; cyto-, cytoplasmic).

FIG. 11: DHA significantly decreases cell viability and inhibits cell growth in human ovarian carcinoma cells by increasing apoptosis, both alone and in combination with carboplatin (CBP). (A) Viability of ovarian epithelial cells after 48 hrs exposure to CBP (0, 1, 10, 50, 100, 500, 1000 μM) in the presence or absence of 1 μM DHA; (B) Cell growth inhibition following exposure to CBP (10 μM) with 1 μM DHA for 0, 24, 48 or 72 hrs; (C) Apoptosis of ovarian cancer cells after exposure to 500 μM CBP with or without 1 μM DHA for 24 hrs, and the corresponding data summary and analysis (*, P<0.001 versus the control, respectively).

FIG. 12: DHA significantly inhibits tumor growth and induces apoptosis alone or in combination with carboplatin (CBP) in mice bearing A2780 and OVCAR-3 xenograft tumors. (A) and (B) Inhibition of tumor growth in mice bearing A2780 or OVCAR-3 xenograft tumors, and the corresponding body weight changes during the treatments; (C) Western-blot analysis of proteins involved in the apoptotic pathway.

FIG. 13: Cartoons of the proposed mechanisms of action of DHA alone and in combination with CBP: (A) The ‘death receptor- and mitochondrion-mediated caspase-dependent apoptotic pathway’ demonstrates how DHA may exert anticancer effects in ovarian cancer cells; (B) Mechanism by which DHA enhances the therapeutic effects of carboplatin.

### DETAILED DESCRIPTION

Embodiments of the invention relate to methods for treating human cancers. Embodiments of this invention may be applied to treat human cancers regardless the p53 status in tumors. For clarity of illustration, the following description will use ART and its various derivatives (DHA, ARM, and ARS) alone or combined with other chemotherapeutic agents such as gemcitabine or carboplatin for treating human hepatoma or ovarian cancer, respectively, as examples. However, as shown in the description below, ART derivatives may function by sensitizing the cancer cells to other therapeutic agents, one of ordinary skill in the art would appreciate that the same approaches can be applied to other combination therapy for treating other cancer types.

ART derivatives may exhibit anticancer potentials. The potential mechanisms underlying this anti-cancer activity may involve induction of apoptosis, selective cytotoxicity of cancer cells, modulation of gene expression, causation of cell cycle arrest, and inhibition of angiogenesis.

Other attractive features that make ART and its derivatives as potential anticancer agents may include low toxicity to the hosts. For instance, DHA may selectively inhibit the growth of Molt-4 lymphoblastoid cells, but to a significantly less extent, i.e., less toxic, to normal human lymphocytes. ARS may inhibit the growth of Kaposi's sarcoma, and this growth inhibition may correlate with induction of apoptosis.

Experimental therapy of hepatoma with artemisinin and its derivatives: in vitro and in vivo activity, chemo-sensitization, and mechanisms of action.

ART and its derivatives selectively inhibit cell growth in human hepatoma cells.

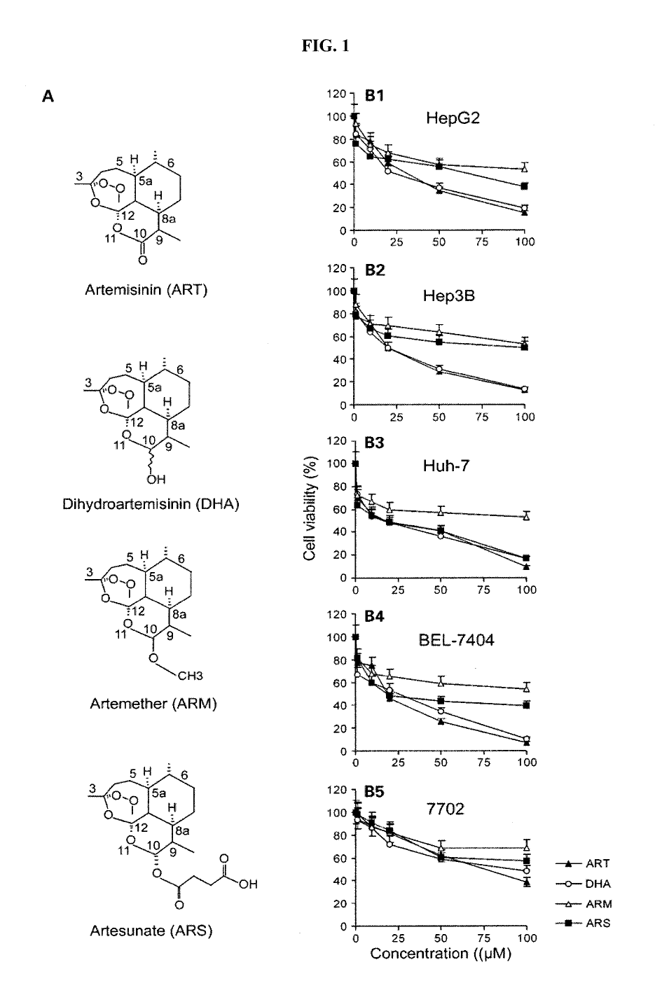

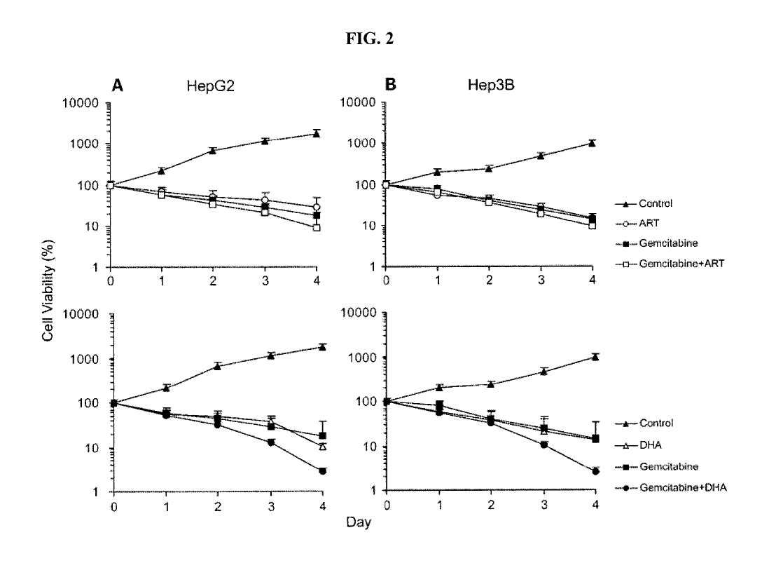

The cytotoxicity of ART and its derivatives (FIG. 1A) are determined against HepG2, Hep3B, BEL-7404, and Huh-7 hepatoma cells as well as 7702 normal human liver cells. The treatment of HepG2 cells with ART or DHA (1-100 μmol/L) results in a significant reduction in cell viability as assessed by the MTT assay, with the percentage of viable cells ranging from 84.7% to 15.5% (P<0.01) after a 48-h exposure (FIG. 1, B1). Similar effects are obtained with Hep3B, Huh-7, and BEL-7404 hepatoma cells (P<0.01; FIG. 1, B2-B4). The concentrations that reduce growth by 20%, 50%, and 80% (IC₂₀, IC₅₀, and IC₈₀) are summarized in Table 1. A comparison of the IC₅₀ values indicates that ART and DHA are the most active compounds, followed by ARS and then ARM (FIG. 1, B1-B4; Table 1). The overall mean IC₅₀ values in the four hepatoma cell lines are 10.8 μmol/L (ART), 10.6 μmol/L (DHA), 21.0 μmol/L (ARS), and 42.3 μmol/L (ARM), respectively. In contrast, the sensitivity of the 7702 cells to the cytotoxic effects of ART and DHA is much lower, with IC₅₀ values ranging from 60.9 to >500 μmol/L (FIG. 1, B5; Table 1), representing a 6- to 16-fold difference in cytotoxicity. These data suggest that ART and its derivatives are cytotoxic to human hepatoma cells, with almost equal efficacy against cancer cells with various p53 statuses, including p53 wild-type, p53 mutant, and p53 null cells, but that these compounds are less cytotoxic to normal human liver cells (FIG. 1, B1-B5).

TABLE 1
Growth-inhibitory activity of ART compounds

| | Inhibitory | | | | |
|---|---|---|---|---|---|
| | concentration | | | | |
| Cell lines | (μmol/L) | ART | DHA | ARM | ARS |
| 7702 | IC₂₀ | 3.6 | 3.8 | 7.9 | 7.3 |
| | IC₅₀ | 60.9 | 167.7 | 492 | >500 |
| | IC₈₀ | >500 | >500 | >500 | >500 |
| HepG2 (p53 | IC₂₀ | 1.3 | 1.2 | 2.6 | 1.3 |
| wild-type) | IC₅₀ | 13.98 | 13.35 | 54.8 | 20.5 |
| | IC₈₀ | 145.1 | 145.8 | >500 | 338.2 |
| Hep3B (p53 | IC₂₀ | 0.97 | 0.96 | 2.4 | 1.5 |
| null) | IC₅₀ | 10.4 | 10.3 | 51.5 | 39.4 |
| | IC₈₀ | 113.3 | 110.7 | >500 | >500 |
| Huh-7 (p53 | IC₂₀ | 0.7 | 0.7 | 1.2 | 0.6 |
| mutant) | IC₅₀ | 8.9 | 9.6 | 31.4 | 9.22 |
| | IC₈₀ | 115.4 | 130.9 | >500 | 146.1 |
| BEL-7404 | IC₂₀ | 0.9 | 0.7 | 2.4 | 1.0 |
| (p53 mutant) | IC₅₀ | 9.9 | 9.3 | 31.78 | 15.0 |
| | IC₈₀ | 107.1 | 129.7 | >500 | 215.4 |

ART and DHA sensitize hepatoma cells to gemcitabine in vitro.

The possible chemo-sensitization effects of ART and DHA are determined in vitro using the MTT assay. As illustrated in FIG. 2A, exposure of HepG2 cells to the two compounds, especially DHA, results in significant growth inhibition. When compared with vehicle-treated cells, HepG2 cells exposed to ART and DHA alone show growth inhibition at as early as 24 h, with 69% and 74% growth inhibition (P<0.05), and with 92% and 93% growth inhibition (P<0.05) at 48 h, and 96% and 97% (P<0.05) at 72 h. Hep3B cells exhibit an almost identical reduction in viability under these conditions (FIG. 2B).

As shown in FIG. 2, the combination of ART with gemcitabine leads to a slight increase in the inhibition of proliferation of hepatoma cells compared with the single agents alone. In both HepG2 and Hep3B cells, the combination of gemcitabine and DHA leads to a statistically significant decrease in cell survival (P<0.05; FIGS. 2A and B, bottom). The increase in inhibition of proliferation by DHA plus gemcitabine compared with gemcitabine alone was 1.2-fold.

ART and DHA induce G1-phase cell cycle arrest in human hepatoma cells.

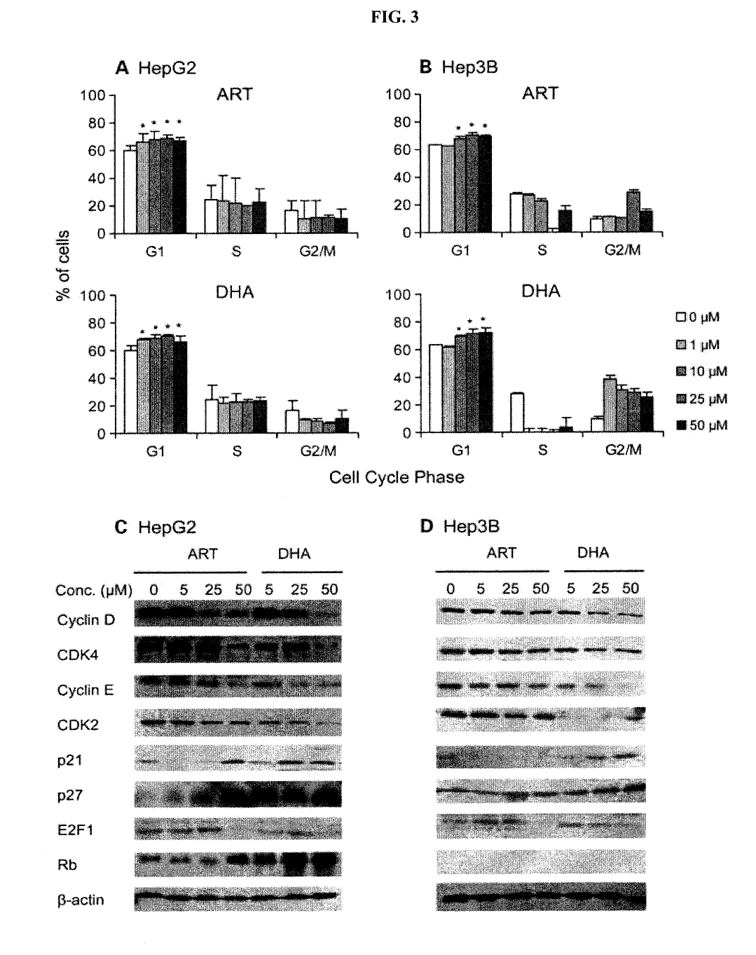

A significant growth-inhibitory effect of ART and DHA on hepatoma cells is observed. To determine whether ART and DHA have any inhibitory effect on cell cycle progression, HepG2 cells are treated with ART. The results show a higher number of cells in the G1 phase at the concentrations used [10 μmol/L (67.41%), 25 won (70.72%), and 50 μmol/L, (69.21%)], respectively, compared with untreated control cells (63.05%; FIG. 3A, top). Similar, but slightly more pronounced, results are obtained when the effect of DHA on HepG2 cells is tested, with even the 10 μmol/L concentration significantly increasing the number of cells in the G1 phase (69.36%, P<0.01), and the higher concentrations leading to greater G1 arrest [25 μmol/L (70.91%, P<0.01) and 50 μmol/L (72.03%, P<0.01); FIG. 3A, bottom].

G1-phase arrest is also observed when the effects of ART and DHA on cell cycle progression of Hep3B are analyzed (P<0.05; FIG. 3B). The lowest concentration of 1 won leads to a modest increase in the number of cells in the G1 phase (66.11%, 68.11%), and higher concentrations of the compounds lead to greater cell cycle arrest [10 μmol/L (67.48%, 69.03%), 25 μmol/L (68.70%, 70.50%), and 50 μmol/L (62.99%, 62.99%), respectively]. DHA shows stronger inhibitory effects on cell cycle progression. These data suggest that inhibition of cell proliferation in both p53 wild-type and p53 null hepatoma cells by ART and DHA is associated with the induction of G1 arrest.

ART and DHA down-regulate cyclins and Cdks and up-regulate Cip1/p21 and Kip1/p27 in human hepatoma cells.

Because Cdks, Cdk inhibitors, and cyclins play essential roles in the regulation of cell cycle progression, the effects of ART and DHA on the expression of these proteins are determined. As shown in FIGS. 3C and D, the effects of DHA are dose dependent and are stronger than those in cells exposed to ART. Treatment with DHA results in a marked reduction in the expression of cyclin D1, cyclin E, Cdk2, and Cdk4 in a dose dependent manner in HepG2 and Hep3B cells. Analysis of the expression of Kip1/p27, Cip1/p21, and E2F1 indicates that DHA causes dose-dependent increases in Kip1/p27 and Cip1/p21 expression and decreased E2F1 expression in HepG2 and HepB3 cells (FIGS. 3C and D). The expression of Rb is also induced by ART and DHA in HepG2 cells (FIG. 3C). These observations suggest that the increases in the levels of Cdk inhibitors may play an important role in the induction of G1 arrest in p53 wild-type and p53 null human hepatoma cells, possibly through their inhibition of Cdk kinase activity.

ART and DHA induce apoptosis in human hepatoma cells.

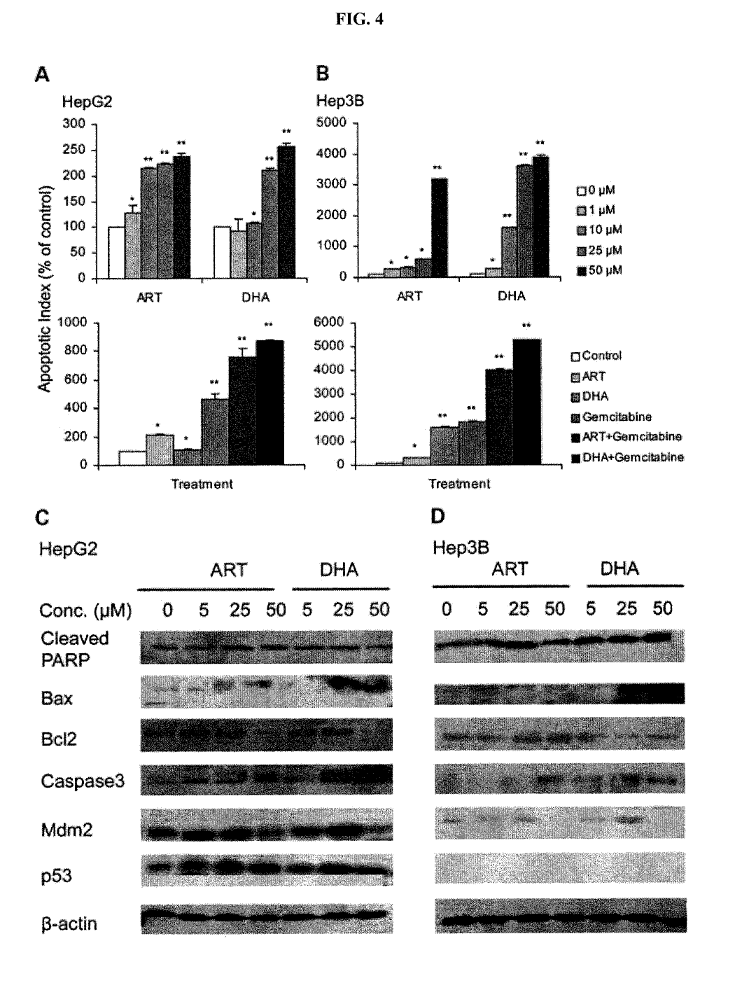

To determine whether the ART- and DHA-induced growth inhibition in hepatoma cells was associated with the induction of apoptosis, HepG2 and Hep3B cells are treated with ART and DHA as described above, and the numbers of apoptotic cells are assessed. Exposure of HepG2 cells to ART for 48 h resulted in a significant dose-dependent increase in apoptotic cells: 0 μmol/L (7%), 1 μmol/L (9%, P<0.05), 10 μmol/L (14.95%, P<0.01), 25 μmol/L (15.45%, P<0.01), and 50 μmol/L (16.45%, P<0.01; FIG. 4A, top). Similar results are obtained when the HepG2 cells are exposed to DHA (FIG. 4A, top).

Gemcitabine is a known inducer of apoptosis in human cancers, including HCC cells, and combination with ART or DHA seems to further increase apoptosis in HepG2 cells (FIG. 4A, bottom). Exposure of Hep3B cells to ART and DHA also results in a significant dose-dependent induction of apoptosis, and the effect of DHA is stronger than that of ART: 0 μmol/L (0.8%), 1 μmol/L (2.25% and 2.15%), 10 μmol/L (2.65%, P<0.05; 12.9%, P<0.01), 25 μmol/L (4.8%, P<0.05; 28.75%, P<0.01), and 50 μmol/L (25.3%, 31.05%, P<0.01; FIG. 4B, top), again indicating that ART and DHA are effective against p53 wild-type and null hepatoma cells. Although apoptosis is induced by all three of the agents alone, it is further increased by the two combinations, especially the combination of DHA and gemcitabine, which improves the efficacy by 2-fold (FIG. 4B, bottom), suggesting that the chemo-sensitizing capacities of ART and DHA maybe associated with induction of apoptosis in the hepatoma cells.

ART and DHA induce changes in the expression of apoptosis related proteins in HepG2 and Hep3B cells.

The proteins of the Bcl-2 family play critical roles in the regulation of apoptosis. Because both ART and DHA induce apoptosis in hepatoma cells, the levels of Bcl-2 and Bax in cells treated with ART and DHA are further determined. HepG2 cells exposed to ART or DHA show a dose-dependent reduction in the level of Bcl-2 protein, with a concomitant increase in the level of Bax, compared with the control cells (FIG. 4C), although DHA exhibited a greater effect on the level of Bax protein than ART.

To define how the apoptotic pathway is activated by ART and DHA, the effects of ART and DHA on the activation of caspase-3 and PARP are determined. Exposure of HepG2 cells to ART and DHA results in a dose-dependent increase in the cleavage of caspase-3 and PARP and ART is less effective than DHA (FIG. 4C). This indicates that the mitochondrial apoptotic pathway is activated preferentially by the compounds.

Caspase-3, an executioner caspase activated by caspase-9, cleaves a broad spectrum of cellular target proteins, including nuclear PARP, leading to a cell death cascade. One of the critical mediators of the mitochondrial apoptotic pathway is p53. Treatment of HepG2 cells with ART and DHA results in a dose-dependent increase in p53 and a decrease in MDM2 (FIG. 4C), suggesting that ART and DHA may induce apoptosis by increasing the level of p53 in HepG2 cells. However, a p53-independent mechanism for an increase in the ratio of Bax/Bcl-2, activation of caspase-3 and the mitochondrial apoptotic pathway, as well as inhibition of MDM2 is also observed in p53 null Hep3B cells (FIG. 4D), suggesting that the ART- and DHA-induced caspase-3 activation may be both p53 dependent and independent.

ART and DHA inhibit tumor growth and have chemo-sensitization effects in vivo.

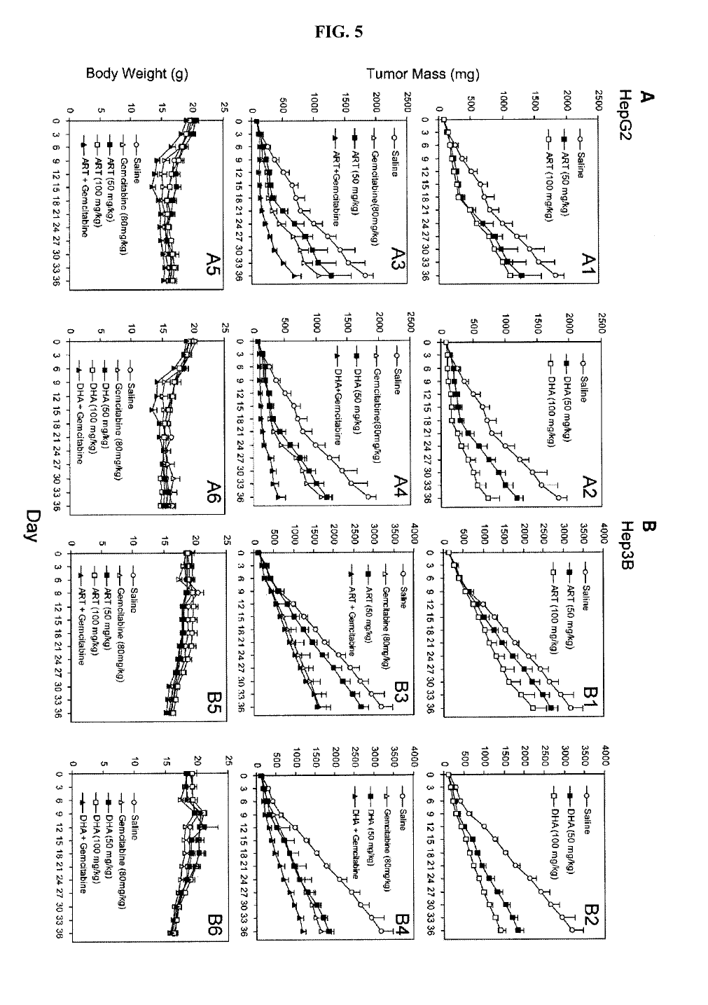

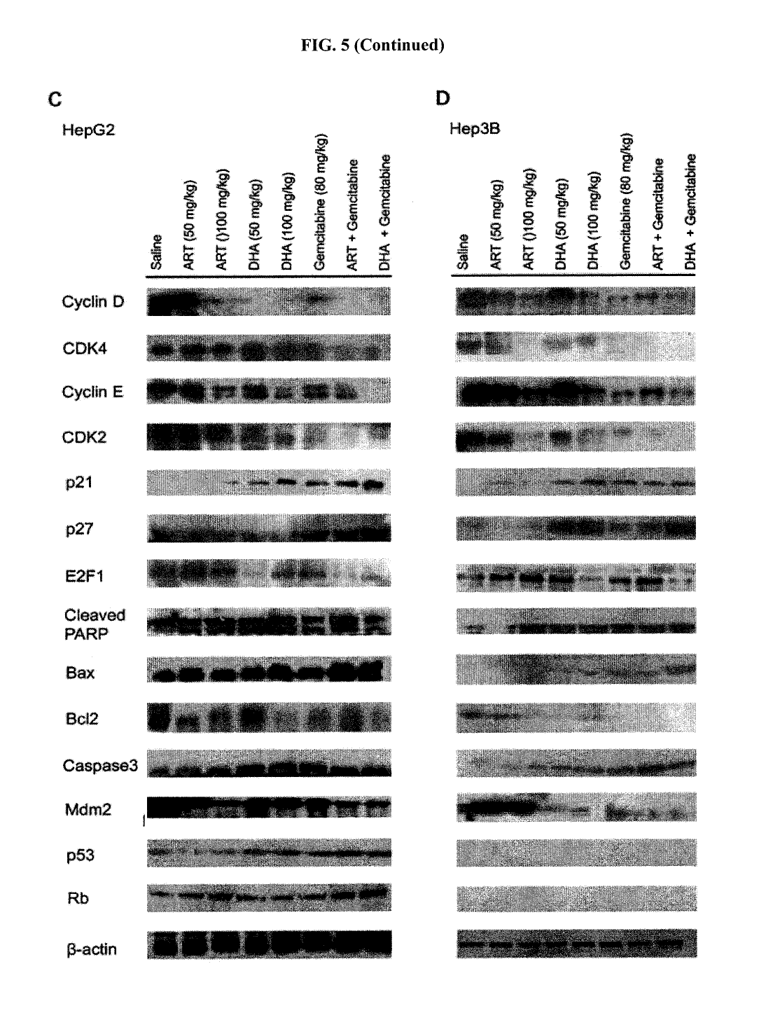

The in vivo antitumor activities of ART and DHA are determined in mouse HepG2 and Hep3B xenograft models. When mean tumor mass reaches 100±40 mg, animals are treated with ART or DHA at oral doses of 50 and 100 mg/kg/d. In the HepG2 xenograft model, both ART and DHA alone show a dose-dependent inhibitory effect on tumor growth (30.0% and 39.4% tumor growth inhibition for ART; 36.1% and 60.6% for DHA; P<0.01; FIGS. 5A1 and A2). Consistent with the in vitro findings, DHA shows greater therapeutic effects in vivo compared with ART.

Because of an increase in anticancer activity following combination treatment with the ART compounds and gemcitabine in vitro, the effects of ART and DHA in combination with gemcitabine are further investigated in vivo. As illustrated in FIG. 5A3, gemcitabine alone decreases tumor growth (34.9% tumor growth inhibition). A simple additive effect is observed for the combination of ART with gemcitabine (62.3% tumor growth inhibition). However, combining DHA with gemcitabine significantly increases the anticancer effect (78.4% tumor growth inhibition; P<0.01; FIG. 5A4), indicating that the combination of DHA and gemcitabine is more effective than ART with gemcitabine. Moreover, based on observations of bodyweight, neither ART nor DHA causes any observable toxic effects when administered alone or in combination with gemcitabine (FIGS. 5A5 and A6).

Similarly, ART shows a slight inhibitory effect on tumor growth in the Hep3B xenograft model (FIG. 5B1), and DHA shows greater, dose-dependent therapeutic effects compared with ART (P<0.01; FIG. 5B2). As illustrated in FIG. 5B3, the combination of ART with gemcitabine shows no statistically significant increase in the inhibition of tumor growth. However, there is a further increase in the antitumor effects when the animals are treated with the combination of DHA and gemcitabine (P<0.01; FIG. 5B4). Neither compound causes any observable toxic effects in this model (FIGS. 5B5 and B6).

ART and DHA modulate the expression of proteins associated with apoptosis and cell cycle regulation in vivo.

To determine whether the changes in expression of proliferation- and apoptosis-related proteins induced by ART and DHA in vitro also occur in vivo, protein expression profiles of HepG2 xenograft tissue samples from animals treated with ART and DHA are determined. The results show a decrease in G1-specific Cdks, cyclin D1, cyclin E, Cdk2, Cdk4, and E2F1 in a dose-dependent manner and an increase in p21 and p27 (FIG. 5C). There are also increases in activated caspase-3, cleaved PARP, Rb, p53, and the ratio of Bax/Bcl-2 and a decrease in MDM2 (FIG. 5C), suggesting that the in vivo antitumor activities of ART and DHA are associated with their capacity to induce G1-phase arrest and apoptosis. Similar protein expression profiles are observed in tumors from the p53 null Hep3B xenograft model (FIG. 5D). Taken together, these data suggest that DHA is effective for suppressing the growth of HepG2 and Hep3B xenograft tumors in nude mice and that the compound can be used in combination with gemcitabine to improve the antitumor effect of treatment.

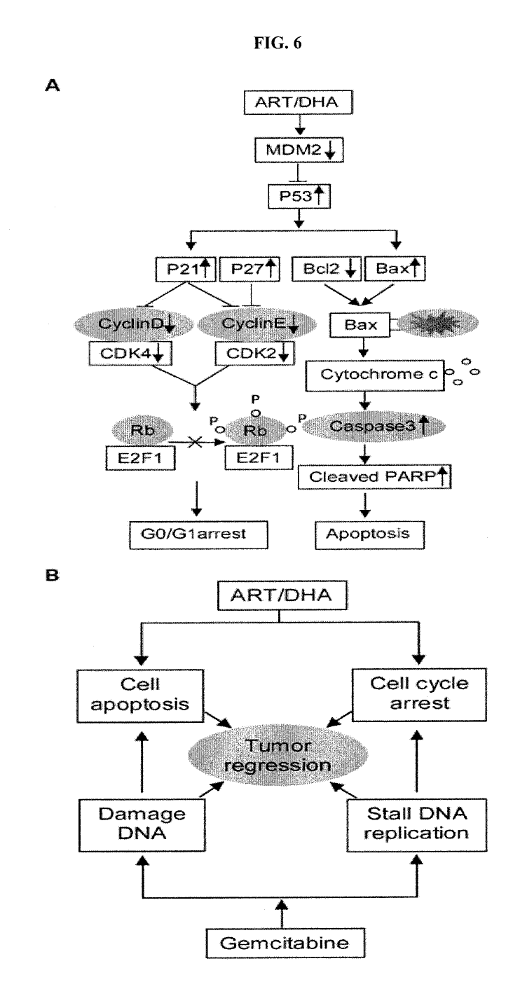

The effects of ART and DHA on various proliferation- and apoptosis-related proteins and the potential mechanism(s) of action of the compounds are summarized in FIG. 6A. The combination of DHA with gemcitabine enhances the induction of apoptosis suggests that combination therapy may improve the antitumor activity of gemcitabine and help define the mechanism by which this occurs (FIG. 6B).

Dihydroartemisinin (DHA) induces apoptosis and sensitizes human ovarian cancer cells to carboplatin therapy.

DHA has the most potent in vitro cytotoxicity in human ovarian cancer cells.

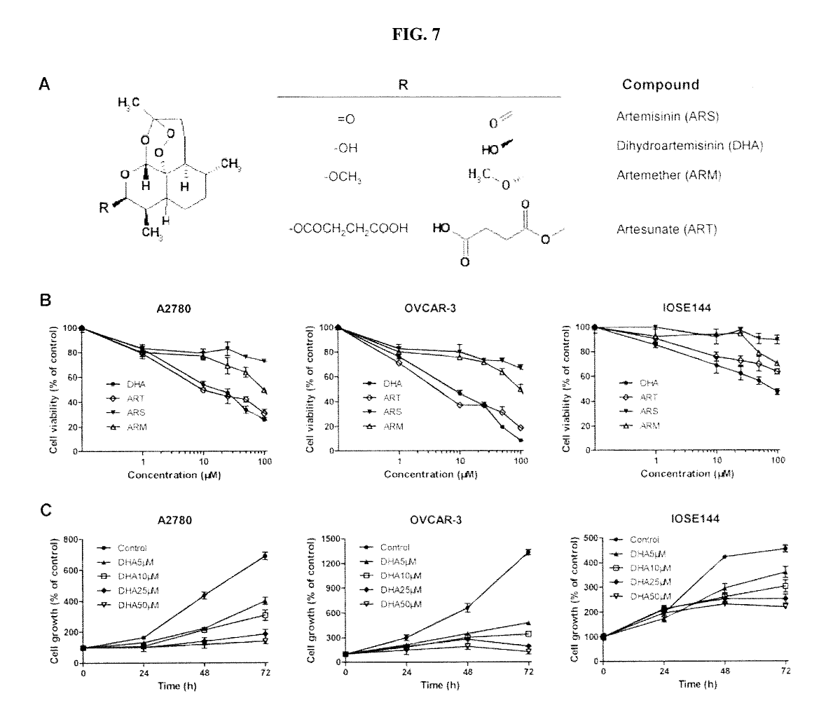

Among the four compounds tested (DHA, ART, ARS, ARM), exposure of ovarian cancer cells to DHA and ART lead to the greatest decreases in cell viability (FIG. 7B, P<0.05). DHA had the lowest IC₂₀, IC₅₀ and IC₈₀ values, and produce the most significant effects on cell survival, inhibiting viability by 24% (1 μM) to 95% (500 μM) (FIG. 7B and Table 2, P<0.05). DHA significantly inhibits the growth of A2780 and OVCAR-3 cells, although the OVCAR-3 cells appear to be more sensitive (FIG. 7C, P<0.05). Immortalized non-tumorigenic ovarian surface epithelial IOSE144 cells are less sensitive to the inhibitory effects of the four drugs than the ovarian carcinoma cells (FIGS. 7B and 7C; Table 2, P<0.05), indicating that the ARS may have selective activity against cancer cells. Taken together, these data suggest that DHA is the most effective ARS, and that it can inhibit the growth of ovarian cancer cells while exerting less potent effects on non-tumorigenic ovarian surface epithelial (OSE) cells.

TABLE 2
Growth inhibitory activity of the four artemisinin
compounds on ovarian epithelial cells

| | Inhibitory | Concentration (μM) | | | |
|---|---|---|---|---|---|
| Cell line | Concentration* | DHA | ART | ARS | ARM |
| A2780 | IC₂₀ | 0.83 | 0.73 | 7.68 | 2.08 |
| | IC₅₀ | 16.45 | 17.60 | >500 | 53.90 |
| | IC₈₀ | 327.63 | 426.16 | >500 | >500 |
| OVCAR-3 | IC₂₀ | 0.56 | 0.46 | 3.93 | 2.08 |
| | IC₅₀ | 6.58 | 6.86 | 342.51 | 38.79 |
| | IC₈₀ | 77.38 | 103.21 | >500 | >500 |
| IOSE144 | IC₂₀ | 1.84 | 5.60 | >500 | 97.95 |
| (Immortalized | IC₅₀ | 106.03 | >500 | >500 | >500 |
| non-tumorigenic | IC₈₀ | >500 | >500 | >500 | >500 |
| OSE cells) | | | | | |

*IC₂₀, IC₅₀, and IC₈₀ are the concentrations of drug that inhibit growth by 20%, 50%, and 80%, respectively, relative to the control.

DHA induces apoptosis in human ovarian cancer cells.

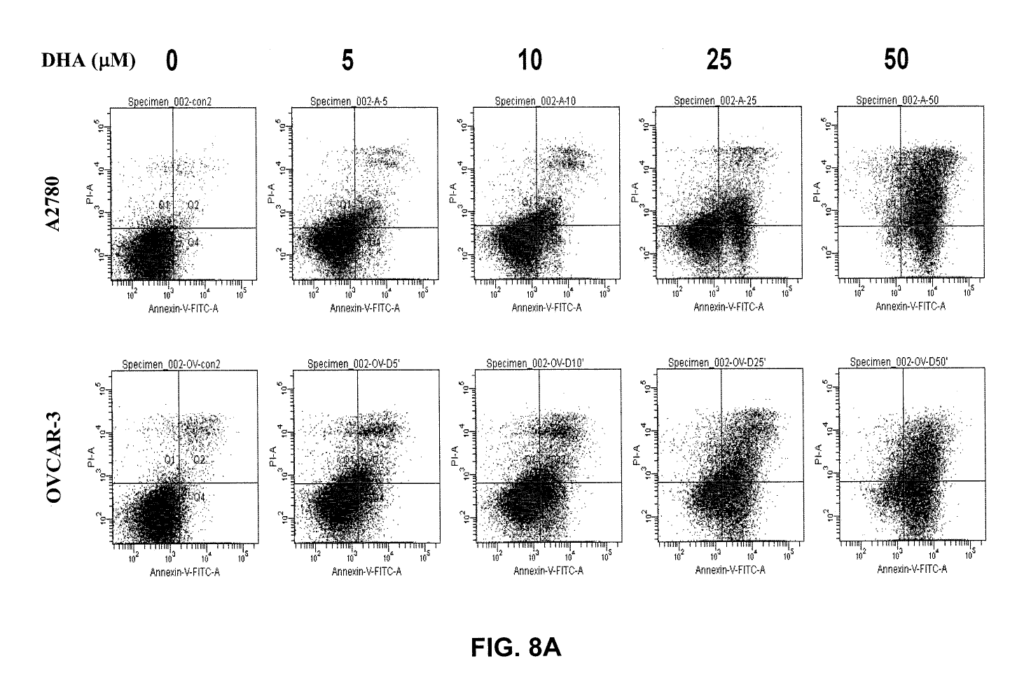

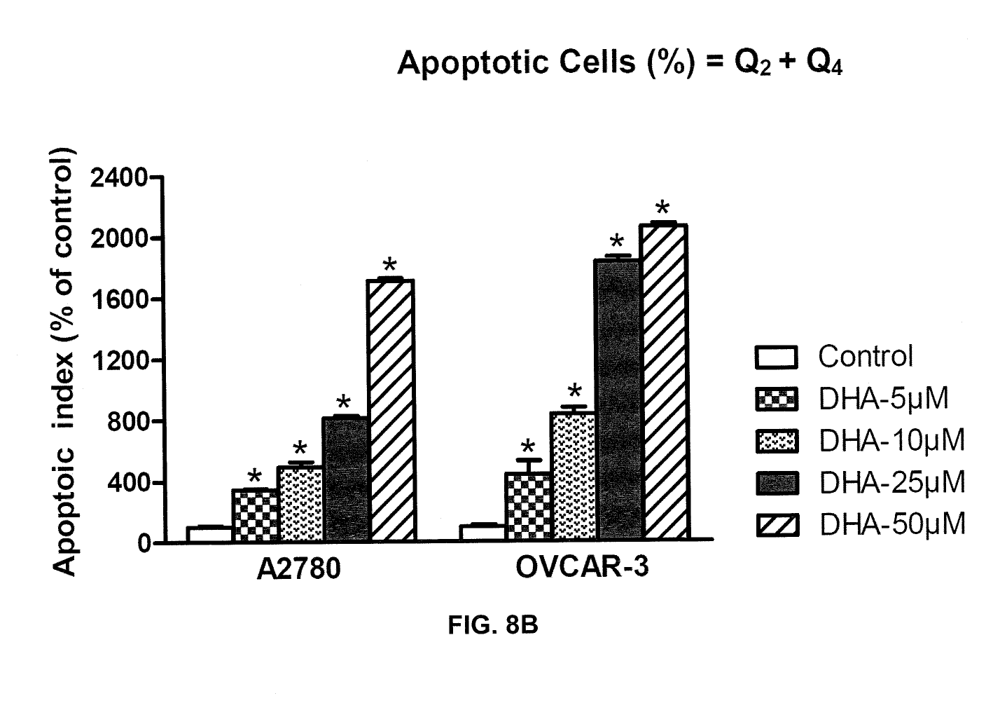

To examine the mechanism responsible for decreasing the viability of the ovarian cancer cells, the effect of DHA on apoptosis is investigated. As demonstrated in FIG. 8A, DHA strongly induces apoptosis in both A2780 and OVCAR-3 cells in a dose-dependent manner. At a concentration of 10 μM, DHA increases apoptosis by about fivefold in A2780 cells, and increases apoptosis by more than eightfold in the more sensitive OVCAR-3 cells (FIG. 8B, P<0.01). These effects are even more pronounced at 25 μM, when apoptosis is increased by more than eightfold in A2780 cells and 18-fold in OVCAR-3 cells (FIG. 8B, P<0.01). When the cells are exposed to 50 μM DHA, they show a 17-fold (A2780) and 22-fold (OVCAR-3) increase in apoptosis (FIG. 8B, P<0.01).

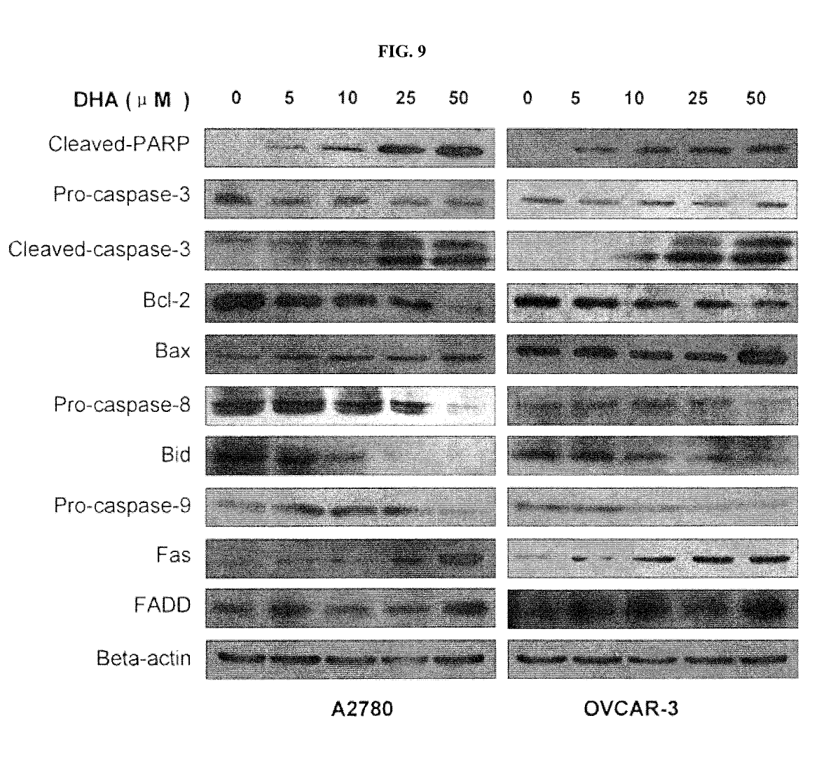

To confirm the effects on apoptosis, the expression of apoptosis-related proteins is evaluated, including PARP, Bax, Bcl-2 and Bid (see FIG. 9). A dose-dependent increase in cleaved-PARP and Bax is observed, while there is a dose-dependent decrease in Bcl-2 and Bid. Moreover, when the expression of pro-caspases-3, 9 and 8 is measured, a dose-dependent cleavage is observed, which is indicative of caspase activation. A dose dependent up-regulation of Fas and its downstream adaptor protein, FADD, which may activate caspase-8, the main enzyme responsible for truncation of Bid is also observed. These data indicate that the activation of caspases plays a major role in the apoptosis induced by DHA and that this apparently occurs via activation of the death receptor pathway.

DHA-induced apoptosis is associated with disruption of the mitochondrial membrane and release of cytochrome c.

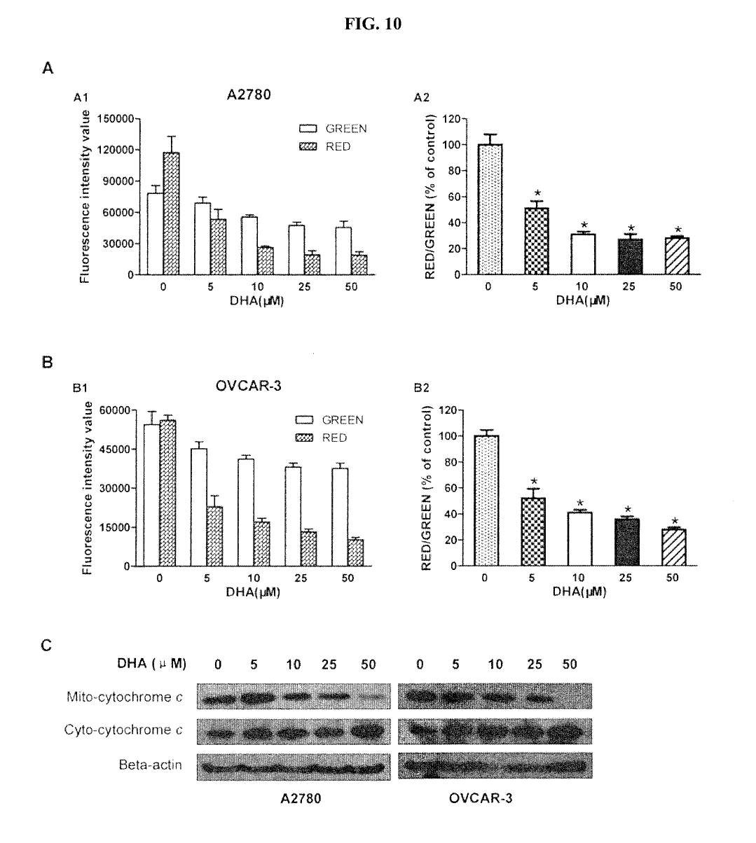

After exposure to different concentrations of DHA, the integrity and potential of the mitochondrial membrane in the A2780 (FIG. 10A) and OVCAR-3 (FIG. 10B) cells are evaluated. These figures demonstrate that exposure to DHA for 24 hrs resulted in a dose dependent dissipation in potential (from 5 to 50 μM) in both A2780 and OVCAR-3 cells, which is indicated by decreased ratios of red (green) fluorescence intensity (FIGS. 10A and B, P<0.05). The mitochondrial cytochrome c levels after exposure to the compound are also determined. The results show a dose-dependent reduction in mitochondrial cytochrome c (FIG. 10C), which is in consistent with the activation of Bid by caspase-8 (FIG. 9). This suggests that DHA may cause the release of cytochrome c from the mitochondria to the cytosol. Together, these results provide evidence that the DHA-induced apoptosis likely occurs through the mitochondrial pathway.

DHA increases the effectiveness of CBP in ovarian cancer cells through an increase in apoptosis.

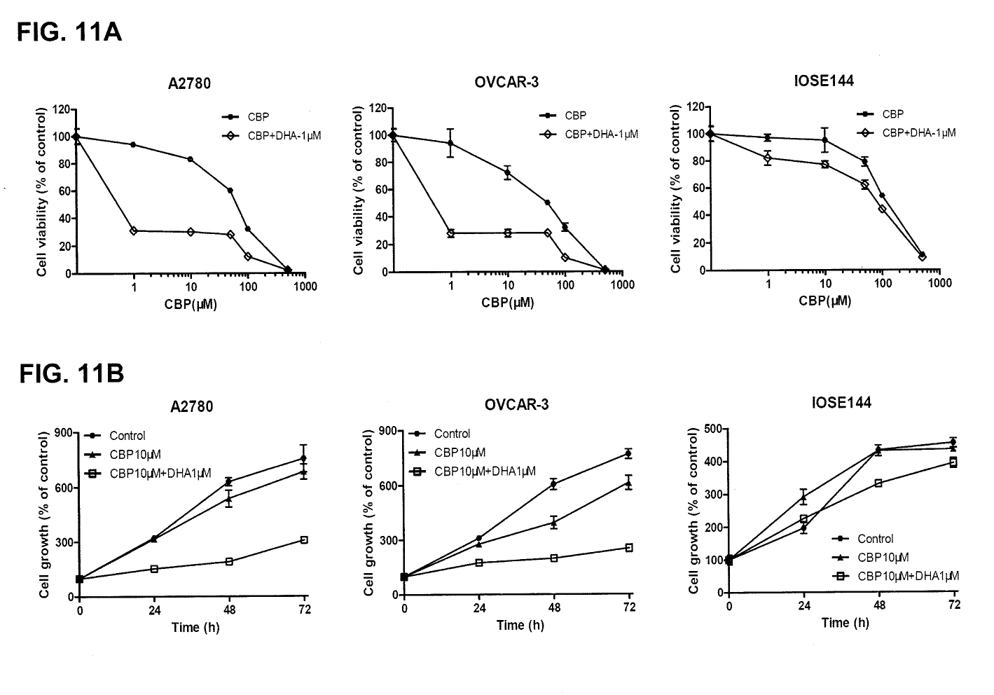

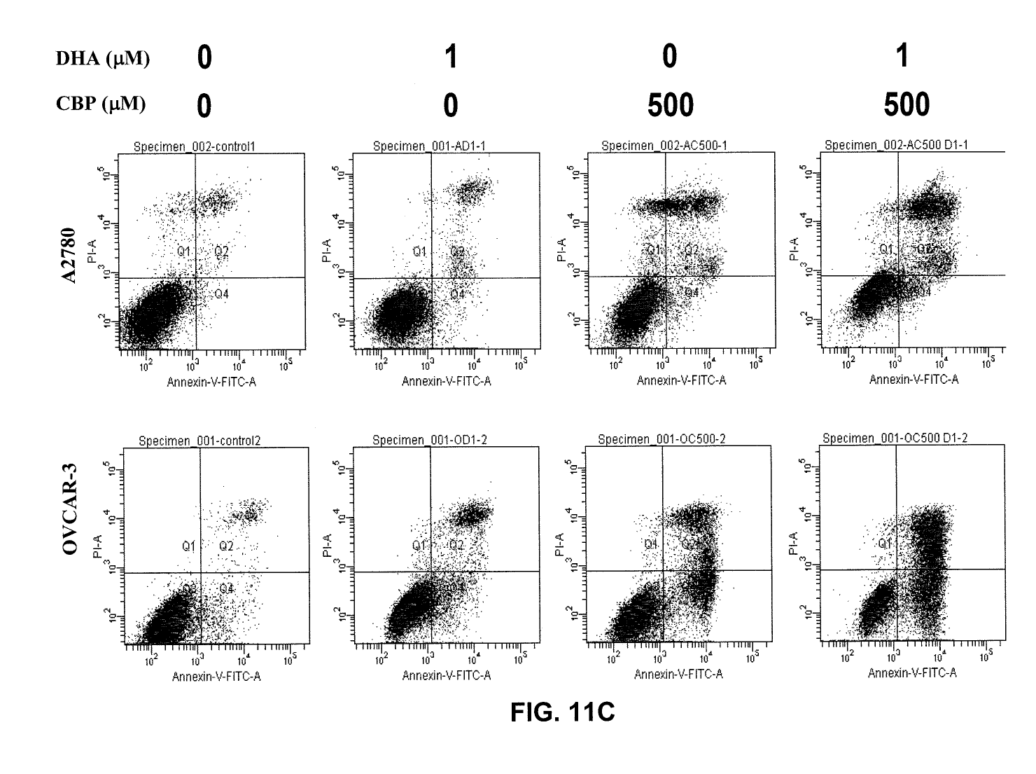

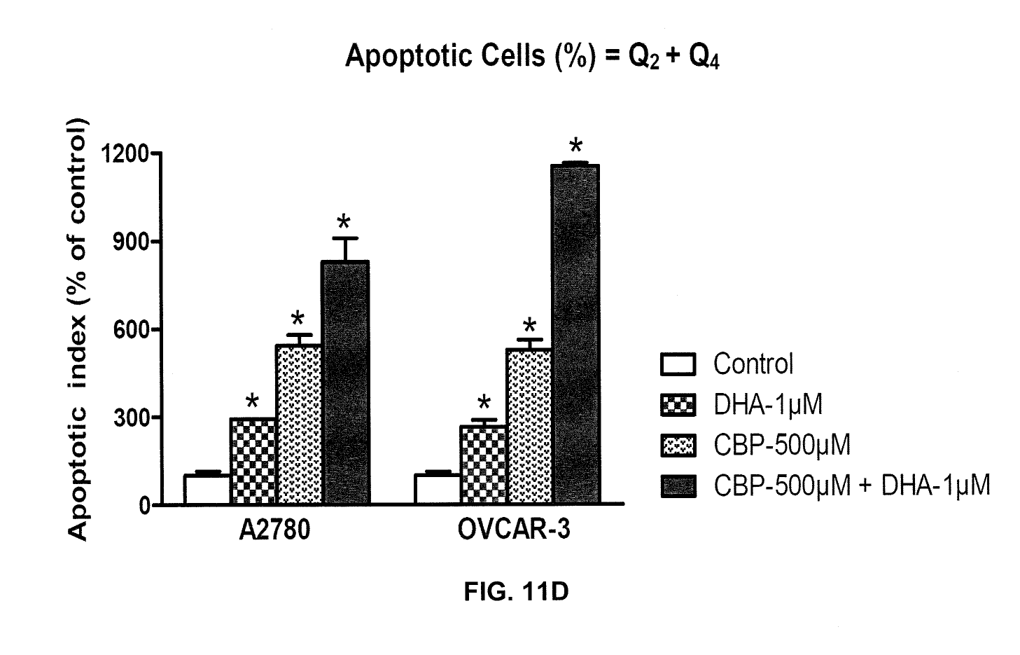

Since combination therapy is a major clinical approach to treatment, the effects of combining DHA with CBP, an agent commonly used to treat ovarian cancer, are investigated. As shown in FIGS. 11A and B, CBP dramatically decreases the viability of ovarian cancer cells when used in combination with DHA. In particular, when cells are exposed to 1 μM CBP and 1 μM DHA, there is a 69% decrease in the viability of A2780 cells, and a 72% decrease in the viability of OVCAR-3 cells. In contrast, IOSE144 cells are much less sensitive to the treatment, with only a 28% decrease in viability when the two compounds are combined at 1 μM each (FIG. 11A, P<0.05).

The effects of the combination on the apoptosis of ovarian cancer cells are also determined. Consistent with the previous results, exposure to a combination of DHA and CBP (24 hrs) appears to induce a synergistic increase in apoptosis in OVCAR-3 cells (FIG. 11C). The rate of apoptosis in cells treated with 1 μM DHA in combination with 500 μM CBP (1155%) is significantly higher than the rate of apoptosis in the cells exposed to 1 μM DHA (266%) or 500 μM CBP (528%) alone, and the rate of apoptosis exceeds the additive effects of the compounds, indicating a potential synergistic effect. However, exposure of A2780 cells to combination treatment leads to an additive effect, rather than synergistic effect. This may be due to their lower sensitivity to the DHA compound (FIG. 11D, P<0.05). A longer exposure to the compound may produce more dramatic effects on apoptosis.

DHA inhibits tumor growth, induces apoptosis and improves CBP therapy in vivo.

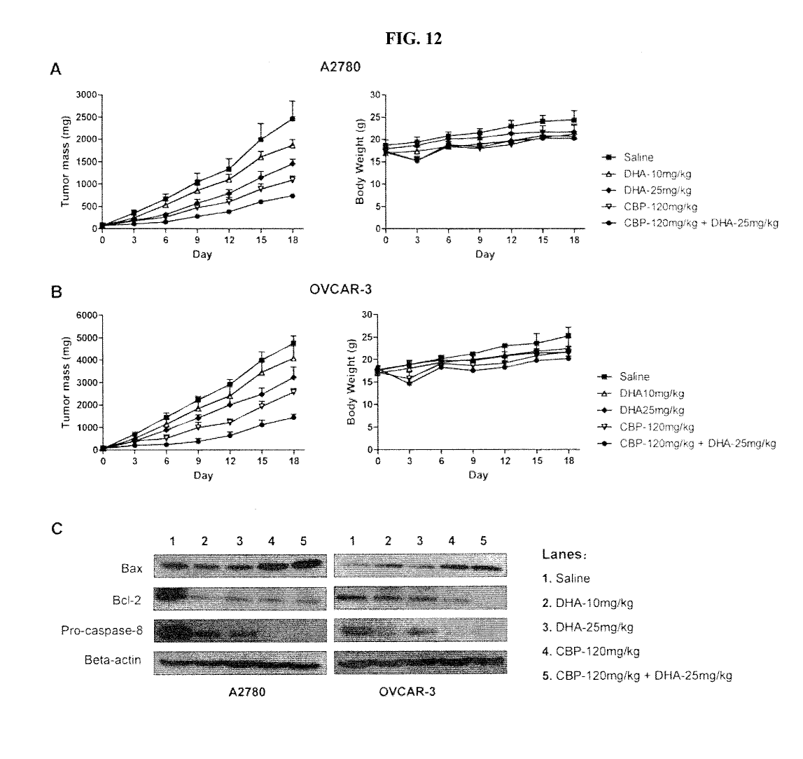

A2780 and OVCAR-3 tumor xenograft models are established to determine whether DHA can exert antitumor effects in vivo. DHA is administered 5 days a week to mice in the treatment groups. CBP is given once on day 0 at a single dose of 120 mg/kg. Therapeutic effects are evaluated by examining tumor growth. As shown in FIGS. 12A1 and B1, DHA (at doses of 10 and 25 mg/kg) results in 24% and 41% tumor growth inhibition (compared to control mice treated with saline) in A2780 xenograft tumor model (FIG. 12A1, P<0.05), and 14% and 37% tumor growth inhibition in the OVCAR-3 model (FIG. 12B1, P<0.05). In the CBP-only group, tumor growth is inhibited by 56% (A2780) and 46% (OVCAR-3) (FIGS. 12A1 and 12B1, P<0.05). Combining the two compounds (25 mg/kg DHA) leads to 70% tumor growth inhibition in both A2780 and OVCAR-3 models (P<0.05). Moreover, based on observation of body weight (FIGS. 12A2 and 12B2), only mice receiving CBP treatment experience slight weight loss. No other host toxicities are observed.

As further validation of the mechanism by which DHA exerts its effects, we assess the in vivo expression of some of the key apoptosis-related proteins mentioned above (FIG. 12C). Consistent with the in vitro findings, a dose-dependent decease in the Bcl-2/Bax ratio and a decrease in pro-caspase-8 are observed, confirming that DHA may exert its effect at least partly by causing apoptosis through the death receptor- and mitochondrion-mediated pathway.

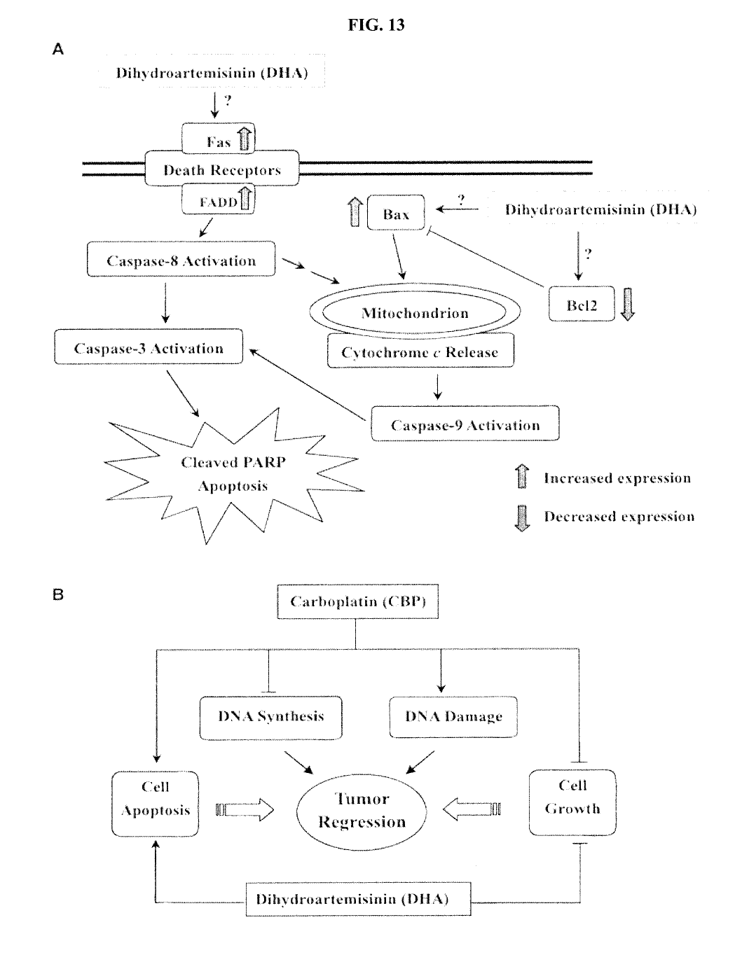

These results suggest that the death receptor and mitochondrion-mediated, caspase-dependent, apoptotic pathway are involved in the activity of DHA (FIG. 13A), and further indicates a possible mechanism by which DHA enhances the therapeutic effects of CBP (FIG. 13B).

### EXAMPLES

### Test Compounds, Chemicals, and Reagents

ART and its derivatives, DHA, ARM, and ARS, are kind gifts of Zhejiang Yiwu Golden Fine Chemical Co. Ltd. Gemzar (gemcitabine) may be purchased from Eli Lilly Co. The 3-(4,5-dimethylthiazol-2-yl)-2,5-diphenyltetrazolium bromide (MTT) and other chemicals used in this study may be of analytic grade and may be purchased from Sigma-Aldrich, Inc. Tween 20 may be purchased from Promega Corp., and the Annexin V-FITC Apoptosis Detection kit may be purchased from BioVision, Inc. The primary antibodies against Bcl-2 (100), Bax (N-20), caspase-3 p20 (N-19), E2F1 (C-20), cyclin D1 (DCS-6), cyclin E (HE12), cyclin-dependent kinase (Cdk) 2 (M2), Cdk4 (H-22), Cip1/p21 (187), Kip1/p27 (C-19), poly(ADP-ribose) polymerase (PARP; H-250), Rb (C-15), MDM2 (SMP14), p53 (Pab1801), glyceraldehyde-3-phosphate dehydrogenase (0411), and β-actin (1-19) may be purchased from Santa Cruz Biotechnology, Inc. The secondary antibodies, horseradish peroxidase-linked anti-mouse immunoglobulin G, anti-goat immunoglobulin G, and anti-rabbit immunoglobulin G, may also be purchased from Santa Cruz Biotechnology. DMEM, RPMI 1640, penicillin, streptomycin, fetal bovine serum, and trypsin/EDTA may be purchased from Life Technologies. The detergent-compatible protein assay kit may be purchased from Bio-Rad and the ECL Plus Western Blotting Detection System may be purchased from Amersham Pharmacia Biotech.

### Cell Culture

Human hepatoma cell lines HepG2, Hep3B, BEL-7404, and Huh-7 and the non-neoplastic human liver cell line 7702 are gifts from the Institute of Biochemistry and Cell Biology, Shanghai Institutes for Biological Sciences, Chinese Academy of Sciences (Shanghai, People's Republic of China). The hepatoma cell lines may be cultured in DMEM supplemented with 10% fetal bovine serum, 100 μg/mL penicillin, and 100 μg/mL streptomycin and maintained in an incubator with a humidified atmosphere of 5% CO₂ at 37° C. The 7702 cells may be cultured with the RPMI 1640 supplemented with 10% fetal bovine serum, 100 μg/mL penicillin, and 100 μg/mL streptomycin under the conditions described above.

Human ovarian IOSE144 (immortalized non-tumorigenic human ovarian surface epithelial cells) and ovarian carcinoma (A2780 and OVCAR-3) cells that may be obtained from the American Type Culture Collection (ATCC, Manassas, Va.) are gifts from Dr. Jing Fang (Institute for Nutritional Sciences, Shanghai, China). All cells may be cultured according to the ATCC instructions. The compounds (ARS, ART, ARM and DHA) may be dissolved in DMSO (<0.1%, final concentration).

### Cell Viability Assay

The effects of ART derivatives on the viability of the aforementioned cells may be determined using the MTT assay. Briefly, 2,000 cells per well were plated in triplicate in 96-well plates. After a 24-h incubation, the cells may be treated with varying concentrations of ART derivatives (0, 1, 5, 10, 25, 50, and 100 μmol/L) for 48 h. The MTT assay may be performed according to the manufacture's instruction and the resultant formazan crystals may be dissolved in DMSO (100 μL). The absorbance may be then recorded at 540 nm. The effects of ART derivatives on cell viability may be assessed by comparing the percent cell viability of the treated cells with the vehicle (DMSO)-treated control cells, which may be arbitrarily assigned at 100% viability. The experiment may be repeated thrice under the same conditions.

In addition, the growth-inhibitory effects of ART and DHA and the effects in combination with gemcitabine in HepG2 and Hep3B cells may also be determined using the MTT assay. Briefly, 700 cells per well may be plated in 96-well culture plates. After a 24-h incubation, the cells may be treated with 10 μmol/L ART, 10 μmol/L DHA, 10 μg/L gemcitabine, 10 μmol/L ART plus 10 μg/L gemcitabine, or 10 μmol/L DHA plus 10 μg/L gemcitabine for various times (0, 24, 48, 72, and 96 h). The results reflect the average of three replicates.

### Cell Cycle Analysis

Cells (2×10⁵) may be treated with ART and DHA (0, 1, 10, 25, and 50 μmol/L) as described above for 48 h. The harvested cells may be re-suspended in 200 μL of cold PBS, to which cold ethanol (600 μl) may be added, and the mixture may then be incubated for 2 h at 4° C. After centrifugation, the pellet may be washed with cold PBS, suspended in 500 μL PBS, and incubated with 50 μL RNase (20 μg/mL final concentration) for 30 min. The cells may be incubated with propidium iodide (50 μg/mL final concentration) for 30 min in the dark. The cell cycle distribution may be determined using a FACSAria instrument (BD Biosciences). The experiment may be repeated thrice under the same conditions.

### Quantification of Apoptotic Cells

ART- and DHA-induced apoptosis alone or in combination with gemcitabine in HepG2 and Hep3B cells or in combination with carboplatin in ovarian cancer cells may be determined by flow cytometry using the Annexin V-FITC Apoptosis Detection kit following the manufacturer's instructions. Briefly, 2×10⁵ cells may be treated with ART and DHA (0, 1, 10, 25, and 50 mol/L) or 10 μg/L gemcitabine for 48 h. The cells may then be harvested, washed in PBS, and incubated with Annexin V and propidium iodide for staining in binding buffer at room temperature for 10 min in the dark. The stained cells may be analyzed using the FACSAria instrument.

### Western Blot Analysis

Whole-cell lysates may be generated with radioimmunoprecipitation assay lysis buffer, and after centrifugation, the supernatant fraction may be collected for immunoblotting. Proteins may be resolved by SDS-PAGE and transferred onto a nitrocellulose membrane. After blocking with 5% nonfat milk in blocking buffer [20 mmol/L TBS (pH 7.5) containing 0.1% Tween 20], the membrane may be incubated with the desired primary antibody for 2 h at room temperature and then incubated with appropriate peroxidase conjugated secondary antibody. The immunoreactive bands may be visualized using the ECL Plus Western Blotting Detection System. The level of (3-actin for each sample may be used as loading control. Tumor tissues may be collected at the termination of the experiment and homogenized using a homogenizer in ice-cold lysis buffer. Supernatants may be collected and used to examine the expression of different proteins by Western blot analysis.

### Mitochondrial Membrane Potential (Δψm) Quantitation

The effect of DHA on mitochondrial membrane potential may be assessed using JC-1 dye. JC-1 is a lipophilic, dual emission fluorescent dye capable of selectively entering mitochondria. Due to the reversible formation of aggregates upon membrane polarization, JC-1 reversibly changes color from green to red at a specific excitation wavelength when membrane potentials increase. It produces red fluorescence (Ex÷550 nm; Em÷600 nm) within the mitochondria (as JC-1-aggregates) proportional to the Δψm. When the Δψm dissipates, JC-1 dye leaks into the cytoplasm (turns into JC-1-monomers) and emits green fluorescence (Ex÷485 nm; Em÷535 nm). JC-1 may be used qualitatively to evaluate the Δψm change according to the pure fluorescence intensity shift between green and red.

To quantify the effect of DHA on mitochondrial membrane potential, A2780 and OVCAR-3 cells may be seeded in 24-well plates (˜2−4×10⁴ cells per well), followed by a 24-hr exposure to DHA at serial concentrations (0, 5, 10, 25, 50 μM). Media may be removed, and cells may then be incubated with RPMI 1640 containing 10 μg/mL of JC-1 dye at 37° C. in the dark for 15 min. Cells may be trypsinized and washed with PBS after the removal of JC-1 dye. Aliquots of 100 μL cell suspensions from the different treatments may be transferred to black 96-well plates. Pure red and green fluorescence intensity may be measured via a fluorescence plate reader (Flexstation II 384, Molecular Devices). Ratios of red/green fluorescence intensity (% of control) may be calculated.

### Hepatoma Xenograft Models

Female athymic nude mice (nu/nu; 4-6 wk of age) may be obtained from Shanghai Slac Laboratory Animal Co. Ltd. All animals may be fed with commercial diet and water ad libitum. The human HCC xenograft models in mice may be as follows. Briefly, HepG2 and Hep3B cells may be re-suspended in serum-free DMEM with Matrigel basement membrane matrix at a 5:1 ratio. The cell suspension may then be injected (7×10⁶ cells; total volume, 0.2 mL) into the left inguinal area of the BALB/c nude mice. The animals may be monitored for activity and physical condition everyday, and the determination of body weight and measurement of tumor mass may be done every 3 d. Tumor mass may be determined by caliper measurement in two perpendicular diameters of the implant and calculated using the formula ½a×b², where a stands for the long diameter and b is the short diameter. The animal use and care protocol is approved by the Institutional Review Board of the Institute for Nutritional Sciences, Chinese Academy of Sciences.

### In Vivo Chemotherapy

Nude mice bearing HepG2 and Hep3B xenografts, randomly divided into various treatment and control groups (five mice per group), may be treated orally with either ART or DHA suspended in 5% sesame oil+95% saline, at a dose of 50 or 100 mg/kg/d, or a combination of ART or DHA with gemcitabine or with saline (as controls). In the mice receiving combination therapy, 80 mg/kg gemcitabine, representing one fifth of the reported most tolerated dose in mice, may be administered i.p. on days 7, 11, and 15 to avoid possible side effects and to illustrate potential chemo-sensitization effects in this combination regimen.

### Mouse Xenograft Model of Ovarian Cancer and Treatment Protocols

Four to six week old female athymic nude mice (BALB/c, nu/nu) may be purchased from Shanghai Experimental Animal Center (Shanghai, China). Animal studies are approved by the Institute for Nutritional Sciences. The tumor xenograft model may be established as follows. Briefly, A2780 and OVCAR-3 cells may be harvested and re-suspended in serum-free RPMI 1640 medium containing 20% (v/v) Matrigel (BD Biosciences, Bedford, Mass., USA). Aliquots of cells (˜5×10⁶ cells/0.2 ml) may be injected subcutaneously into the left inguinal area of the mice. The tumor growth and body weight of the mice may be monitored every other day. Tumor mass may be determined. Mice bearing palpable tumors (˜70 mg) may be randomly divided into treatment and control groups n=5 mice/group). CBP may be dissolved in saline, and DHA may be dissolved in cremophor EL:ethanol:saline (5:5:90, v/v/v). DHA may be administered via i.p. injection at doses of 10 and 25 mg/kg/5 days/week for 3 weeks) alone or combined with CBP (at a single dose of 120 mg/kg, once on day 0). The control group receives saline only. Mice may be killed on day 18. Tumors may be carefully excised, trimmed of extraneous fat or connective tissue, and homogenized in RIPA buffer (100 mg tumor tissue/1 ml RIPA) and prepared for immunoblotting analysis as described above.

### Statistical Analysis

The experimental data may be expressed as mean and SD, and the statistical significance of differences between control and treated groups may be determined by the paired t test or ANOVA.

Advantages of embodiments of the invention may include one or more of the following. Embodiments of the invention use ART or its derivative to sensitize human cancer cells to the anti-tumor activity of the conventional chemotherapeutic drugs. Thus, lower doses may be used to achieve maximum efficacy of treatment with minimum undesired side effects often associated with traditional chemotherapy.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A method of treating cancer in a mammal, comprising administering to the mammal a therapeutically effective amount of artemisinin (ART) or its derivative or in combination with a chemotherapeutic agent.

2. The method of claim 1, wherein the mammal is a human.

3. The method of claim 2, wherein the chemotherapeutic agent is gemcitabine.

4. The method of claim 3, wherein the cancer is liver cancer.

5. The method of claim 2, wherein the chemotherapeutic agent is carboplatin.

6. The method of claim 5, wherein the cancer is ovarian cancer.

7. The method of claim 1, wherein artemisinin (ART) or its derivative is administered orally.

8. The method of claim 1, the artemisinin derivative is dihydroartemisinin (DHA), artemether (ARM), or artesunate (ARS).

9. A method for inhibiting tumor cell proliferation, comprising contacting a tumor cell with artemisinin (ART) or its derivative in an amount effective to inhibit tumor cell proliferation or in combination with a chemotherapeutic agent.

10. The method of claim 9, wherein the chemotherapeutic agent is gemcitabine.

11. The method of claim 10, wherein the cancer is liver cancer.

12. The method of claim 9, wherein the chemotherapeutic agent is carboplatin.

13. The method of claim 12, wherein the cancer is ovarian cancer.

14. The method of claim 9, the artemisinin derivative is dihydroartemisinin (DHA), artemether (ARM), or artesunate (ARS).
